(19) United States
(12) Patent Application Publication
(10) Pub. No.: US 2003/0181863 A1
(43) Pub. Date: Sep. 25, 2003

(54) Microneedle adapter

(76) Inventors: Donald E. Ackley, Cardiff, CA (US); Shawn Davis, Smyrna, GA (US); Thomas Jackson, La Jolla, CA (US)

Correspondence Address: ROPES & GRAY LLP, ONE INTERNATIONAL PLACE, BOSTON, MA, 02110-2624, US

(21) Appl. No.: 10/412,384

(22) Filed: Apr. 11, 2003

Related U.S. Application Data
(63) Continuation-in-part of application No. PCT/US01/46845, filed on Nov. 8, 2001.
(60) Provisional application No. 60/247,571, filed on Nov. 9, 2000.

(51) Int. Cl. A61M 5/24

(52) U.S. Cl. 604/201; 604/205

(57) ABSTRACT

The present invention relates to an adapter for the transport of fluids with a microneedle device. The adapter can receive a syringe, for example, that is used to transport a fluid through the adapter for injection into a patient using the microneedle device. The adapter can include a seal through which a syringe needle is inserted to deliver fluid from the syringe into a fluid cavity in the adapter.

### CROSS-REFERENCE TO RELATED APPLICATIONS

This application is a continuation in part of International Application No. PCT/US01/46845, filed Nov. 8, 2001, which claimed the benefit of U.S. application Ser. No. 09/922,656, filed Nov. 6, 2001, which claimed the benefit of U.S. Provisional Application Serial No. 60/247,571, filed Nov. 9, 2000, the specifications of each of which are incorporated by reference herein in their entirety. International Application No. PCT/US01/46845 was published under PCT Article 21(2) in English.

### BACKGROUND OF THE INVENTION

Syringes have traditionally been used for the transdermal introduction of medication. However this technique has a number of disadvantages including local damage to the skin and associated pain, bleeding and the risks for transmission of disease or infection.

Infusion sets are also commonly used in connection with miniature pumps for insulin delivery as well as for delivering other drugs or fluids. They come in multiple styles with shorter needles for penetration perpendicular to the skin and longer needles for penetration into the skin at a shallow angle.

Alternatively, microneedle devices have been developed to provide for transdermal delivery or removal of fluids without many of the risks associated with standard syringes. Such devices use arrays of small diameter needles that each deliver relatively small flow rates of fluid across or into a biological barrier, which together with the fluid delivered by other needles in the array, provide a clinically useful alternative to standard syringes.

A continuing need exists, however, for further improvement in devices for fluid transfer to improve safety, convenience and manufacturability.

### SUMMARY OF THE INVENTION

The present invention relates to devices and methods involving the use of syringes with microneedles to provide for the transport of fluids across barriers such as the skin. It is desirable to employ a standard syringe to withdraw an injectable fluid from a vial, remove the air from the needle, but inject the fluid through a microneedle or microneedle array. The invention thus involves an adapter that transfers the fluid in the syringe into a microneedle array for delivery. A preferred embodiment includes an adapter having a microneedle array and a port that receives a syringe to provide fluid communication between the syringe and the microneedle array. In this embodiment, the fluid pressure resulting from the use of the syringe is used to transfer the fluid to the adapter and inject the fluid in a single step. Another preferred embodiment involves a two step process in which the fluid is transferred to the adapter and subsequently delivered.

The present invention provides an adapter having a seal or septum in which a syringe needle can be inserted to couple the syringe to a microneedle device. A preferred embodiment of the adapter uses a fluid coupler through which fluid flows upon exiting the syringe needle. The fluid coupler has a size and shape that minimizes the amount of fluid retained in the adapter after use. In a preferred embodiment, a plurality of small diameter needles extend from a distal surface of the adapter, each needle being in fluid communication with a fluid cavity in the coupler between the seal and the proximal ends of the needles.

In a preferred embodiment, the needles are formed integrally with a distal membrane or cap. The membrane covers a distal surface of the fluid coupler having one or more ports through which fluid flows between the cavity and the array. The coupler distal end can have a plurality of channels through which fluid flows. These channels can also be enclosed by the membrane with which the needle array is formed. This membrane or array member can be flexible such that it bends or bows distally upon application of fluid pressure as fluid enters the adapter cavity. The member is displaced by no more than 0.2 mm so that the volume of the cavity expands by a known volume. Pins on the proximal side of the array member can extend into holes on the distal face of the coupler to provide alignment of the needles with the ports or channels on the coupler.

In a preferred embodiment of the invention, an adapter suitable for use with an infusion procedure can be used in which a needle can be inserted into the adapter at a shallow angle. In this embodiment, the external shape of the adapter housing accommodates mounting on a patient's skin such as the forearm.

In some embodiments, microneedles have lengths in the range of 1 micron to 500 microns. In certain embodiments, one or more microneedles can have a length of at least about 500 microns (e.g., at least about 600 microns, at least about 700 microns, at least about 800 microns, at least about 900 microns) and at most about 1500 microns (e.g., at most about 1400 microns, at most about 1300 microns, at most about 1200 microns, at most about 1000 microns), such as from about 800 microns to about 1100 microns (e.g., from about 900 microns to about 1000 microns, from about 930 microns to about 970 microns, about 950 microns).

The microneedles can have an outer diameter of about 10 micrometers to about 100 micrometers.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 illustrates a microneedle adapter for a syringe in accordance with the invention.

FIGS. 2 and 3 show perspective views of the adapter of FIG. 1.

FIG. 4 illustrates an array plate of the adapter during an injection procedure.

FIG. 5 illustrates a fluid path through the fluid coupler.

FIG. 6 is an enlarged cross-sectional view of the coupler.

FIG. 7 is an infusion adapter in accordance with the invention.

FIG. 8 is another preferred embodiment of an adapter in accordance with the invention.

### DETAILED DESCRIPTION OF THE INVENTION

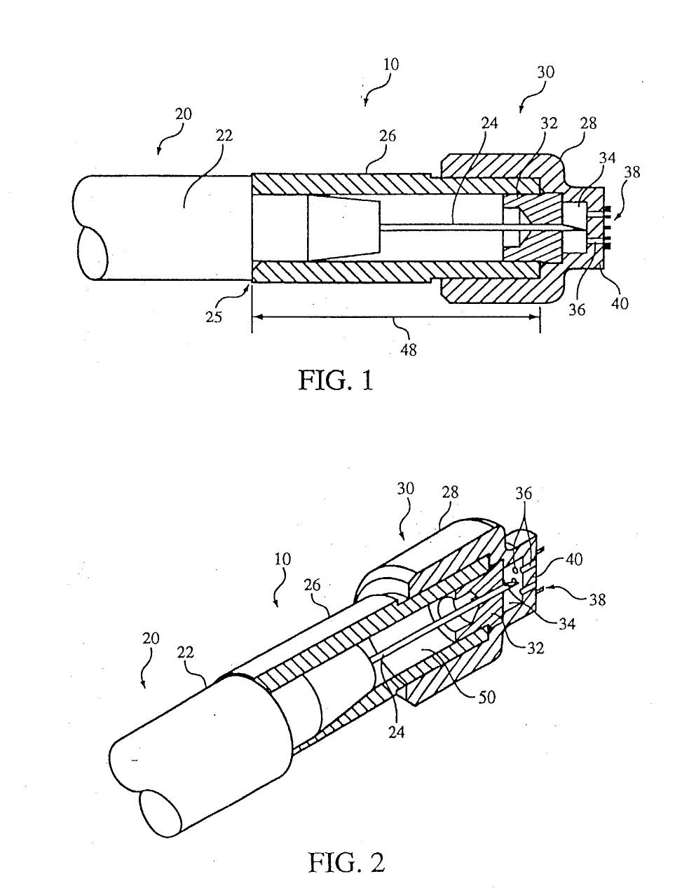

FIGS. 1, 2 and 3 illustrate an adapter which is identified generally as 10. The adapter 10 can receive the distal portion of a syringe 20 having a syringe body 22 and a needle 24. The adapter 10 includes a microneedle array 38, that can allow the injection of fluids into an injection site. The adapter 10 can include a housing 30 having an inner sleeve 26 having a proximal port 25 and an outer sleeve 28. The inner sleeve 26 can have a length 48 wherein the length 48 is variable depending on the length of the needle 24. The inner sleeve 26 and the outer sleeve 28 can each be made separately, or they can be made from a single molded piece of material. The inner sleeve 26 and the outer sleeve 28 can be joined using an adhesive or mechanical joint.

The adapter 10 can include a port 25, located in a proximal end of the adapter and can also include a distal end 40. The adapter distal end 40 can be formed integrally with the outer sleeve 28. The adapter 10 can also include a seal or septum 32 which can be mounted to the inner sleeve 26, the outer sleeve 28 or a portion of both. The septum 32 can allow for centering of the needle 24 within the adapter 10, when the adapter 10 is placed onto a syringe. The septum 32 has a distal surface that partially encloses a fluid cavity 34 having a small volume to minimize dead space for a fluid remaining within the adapter after injection of the fluid into an injection site. The fluid cavity 34 can be formed between the septum 32 and the distal end 40. The small volume of the fluid cavity 34 minimized residual fluid. The range of volume of the cavity is from 5 to 25 microliters.

The distal end 40 can include fluid channels 36 and can include a microneedle array 38. The fluid channels 36 can be in fluid communication with the microneedle array 38 and can also be aligned with the needles in the microneedle array 38. The fluid channels 36 can have a circular cross section as shown in FIG. 2. There can be between one and nine fluid channels 36 located in the distal end 40. The fluid channels can be formed and/or connected using laser drilling. The fluid channels can also be formed by roughening the surface of the distal end, for example.

The microneedle devices of the present invention can be made in accordance with the procedures described in U.S. application Ser. No. 09/095,221 filed on Jun. 10, 1998, U.S. application Ser. No. 09/316,229 filed on May 21, 1999 and in International Application No. PCT/US99/13226 filed on Jun. 10, 1999, the entire contents of all the above applications being incorporated herein by reference.

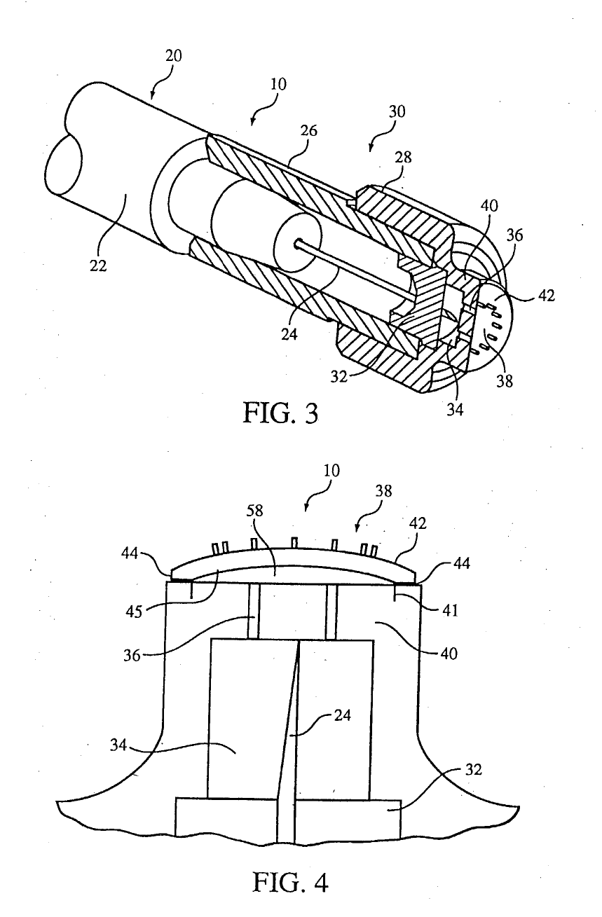

The microneedles 38 can extend from an array member 42 as shown in FIG. 3. The array member 42 forms a base of the microneedle array 38. The microneedles are in fluid communication with the fluid channels 36 of the distal end 40. The needles of the array 38 can have an aperture size of between 10 and 200 microns. The radius of the distal end 40 and the array member 42 can be between 1 mm and 3 mm, preferably either 1.5 mm or 2 mm. The needles 38 can be in a single ring, or a plurality of rings having different radii. The array member 42 can be formed of the same material as the microneedles of the array 38. The array member 42 can be attached to the distal end 40 with an adhesive. The adhesive can form a ring encompassing the outer edge of the array member 42 and the outer edge of the adapter distal end 40. The thickness of the adhesive can be between approximately 5 to 125 micrometers.

To utilize the adapter 10 with a syringe 20, the syringe 20 is filled from a vial using the needle 24. The adapter 10 can then be placed over the needle 24 which penetrates the septum 32 of the adapter 10. The septum 32 can help to guide the needle 24 into the proper position within the adapter 10. The microneedle array 38 of the adapter 10 can then be pressed against an injection site such that fluid penetrates the injection site. The injection site can be skin, for example, and the microneedle array 38 can penetrate the stratum corneum of the skin. The fluid can be injected from the syringe 20 through the needle 24 into the fluid cavity 34 and through the fluid channels 36. The fluid channels 36 transfer the fluid into an injection site through the microneedle array 38.

FIG. 4 illustrates an adapter 10 during an injection procedure. During injection, fluid from the syringe 20 can travel through the needle 24 and into the fluid cavity 34. In turn, the fluid can travel from the fluid cavity 34, into the fluid channels 36. The array member 42 can be formed of a flexible material such that during injection, fluid traveling through the fluid channels 36 causes the member 42 to bend or bow outwards in the distal direction, prior to exiting the microneedle array. Bowing of the array member 42 can be achieved because the array member 42 includes a bonding material 44 around its outer circumference which secures the array member 42 to the distal end 40 only at the bonding site. Pins 41 can be used that extend from a proximal surface 45 of member 42 into holes on the distal surface of distal end 40 to align the needles 38 with the ports or channels 36 during assembly. Alternatively, the cavity can also include an elastic expandable membrane that enlarges the cavity during transfer of fluid but contracts to minimize retained fluid volume after injection. Therefore, the inner portion of the array member 42 is moveable. Fluid from the fluid channel 36 can thereby enter a space 58 located between the distal end 40 and the array member 42. Fluid within the space 58 can then be transferred to an injection site through the microneedle array 38.

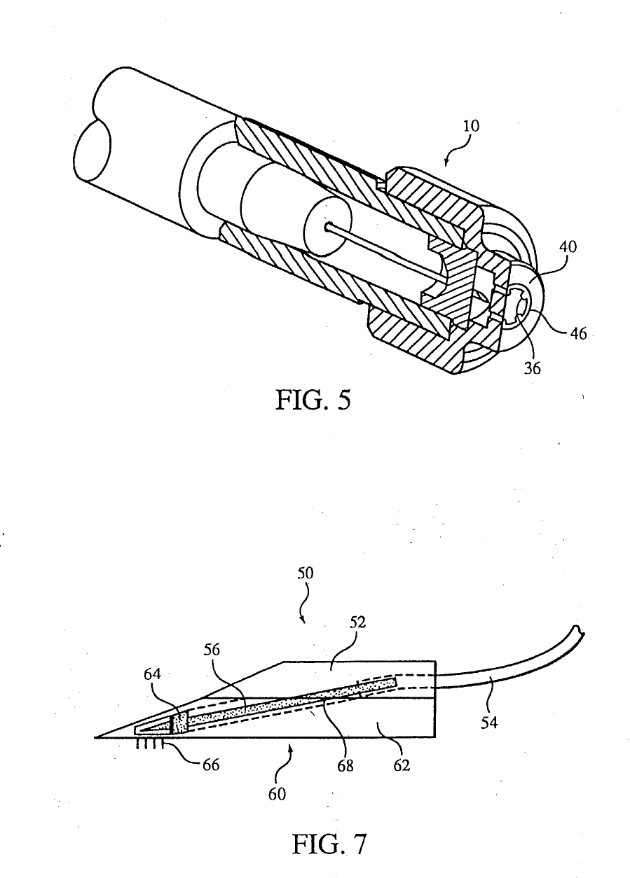

FIG. 5 illustrates an alternate arrangement of the fluid channels 36. The fluid channels 36 can be connected to a fluid chamber 46. The fluid chamber 46 can be formed as a groove within the distal end 40 and can collect fluid from the fluid channels 36.

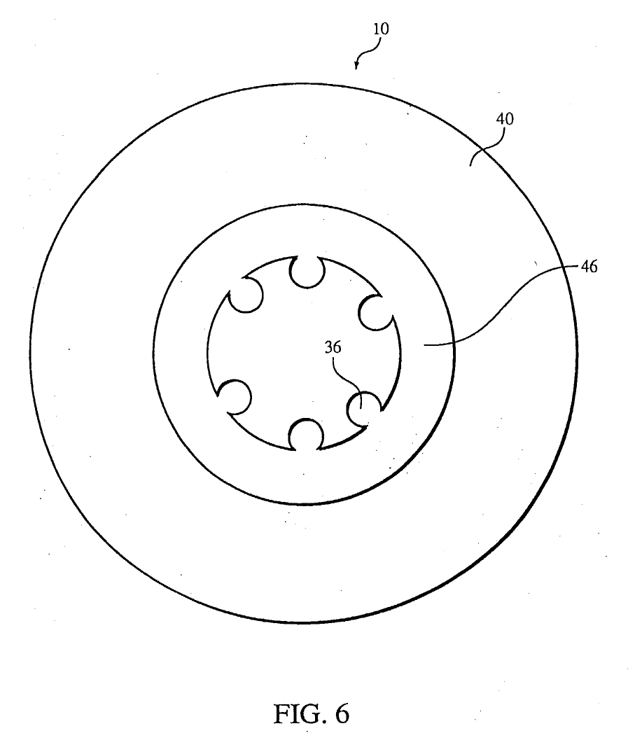

FIG. 6 illustrates the connection between the fluid channels 36 and the fluid chamber 46. The fluid channels 36 are in fluid communication with the fluid transfer chamber 46. In this arrangement, during an injection procedure, fluid from the fluid cavity 34 can travel through the fluid channels 36 and into the fluid chamber 46. From the fluid chamber 46, the fluid can travel through the microneedle array 48 mounted over the fluid chamber 46.

During an injection procedure, after the microneedle array 38 has been placed into an injection site and fluid from the syringe 20 has been injected into the fluid site, the syringe 20 and needle 24 can be removed from the adapter 10. While the adapter 10 is maintained within the injection site, such an arrangement can allow for subsequent needles 24 to be inserted into the adapter. Subsequent doses of fluid can be delivered to the injection site in this manner, thereby minimizing the skin penetration to only the area where the microneedle array is attached to the injection site. This minimizes the number of piercing sites necessary for the delivery of a fluid in a repetitive manner.

An adapter having a microneedle array can be used in conjunction with an infusion set, whereby the adapter can be shaped to match with a body of an infusion device. FIG. 7 illustrates an infusion set 50 having an infusion set body 52 and an adapter 60. The infusion set body 52 can include a fluid conduit 54 and a needle 56, whereby the needle 56 connects to the fluid conduit 54. The fluid conduit can also be attached to a pump, such as a micropump, having a fluid source. The adapter 60 can include an aperture 68 formed at a shallow angle relative to a body 62 of the adapter 60. A shallow angle is defined as an angle of between 0° and 45° relative to a long axis of the housing or body 62. The adapter 60 can also include a septum 64 and a microneedle array 66. Also, the adapter 60 can have a microneedle array 66 formed at the end of a housing 62 having an angle which is non-parallel to the long axis of the housing.

To use the adapter 60 with an infusion set body 52, rather than inserting the needle 56 of the infusion set body 52 into an injection site by pushing at an angle nearly parallel to the surface of the skin, the adapter 60 can be inserted over the needle at a shallow angle such that the needle 56 enters the aperture 68 of the adapter 60. The entire infusion set 50 which includes the infusion set body 52 and the adapter 60 can be pushed perpendicular to an insertion site to insert the microneedle array 66.

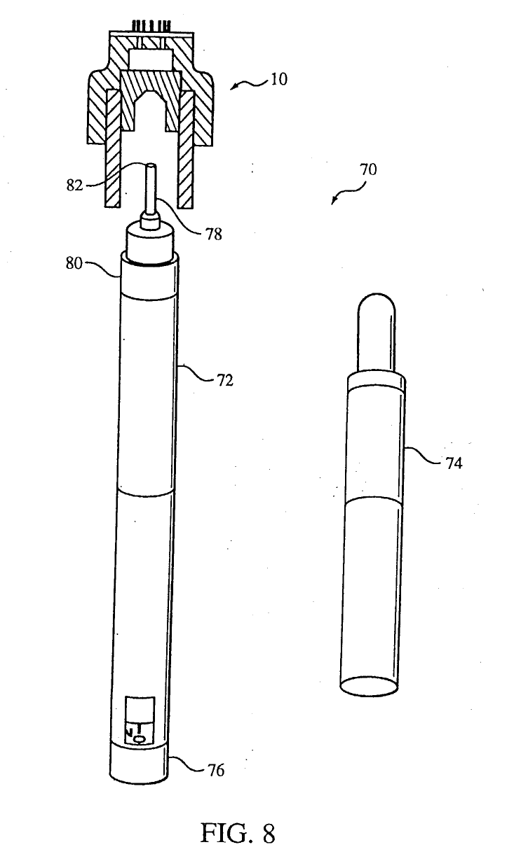

The adapter 10 can also be used in conjunction with a medication delivery pen 70, as is illustrated in FIG. 8. The medication delivery pen can include an insulin pen as is commonly used by Type I diabetics. The medication delivery pen 70 can include a cap 74, a housing 72, a spring-loaded plunger 76, and can also include a medication cartridge housed within the housing 72 of the delivery pen 70. The mediation delivery pen 70 can also include a needle 78 which screws or snaps onto housing 72, and comprises a proximal end 80 which punches a septum at the end of the medication cartridge to allow drug to flow. Needle 78 also comprises a sharpened distal end 82 for insertion into skin, and can be disposable. The adapter 10 can be placed over needle 78 of pen 70 in such manner as to allow drug to flow through needle 78 and out through the microneedles of adapter 10. Alternatively, adapter 10 may incorporate screw threads or a snap-on feature such that it interfaces directly with housing 72 as part of medication delivery pen 70.

While this invention has been particularly shown and described with references to preferred embodiments thereof, it will be understood by those skilled in the art that various changes in form and details may be made therein without departing from the scope of the invention encompassed by the appended claims.

What is claimed is:

1. An adapter for a fluid transport comprising:
a housing having a proximal end and a distal end, the proximal end having an aperture;
a fluid cavity within the housing;
a fluid pathway within the housing; and
a microneedle device mounted to the housing, the microneedle device being in fluid communication with the fluid pathway.

2. The adapter of claim 1 further comprising a septum within the housing.

3. The adapter of claim 1 further comprising a plurality of fluid channels.

4. The adapter of claim 1 wherein the microneedle device is attached to the housing with a fluid coupler.

5. The adapter of claim 4 wherein the microneedle device comprises an array of microneedles mounted to the fluid coupler at the distal end.

6. The adapter of claim 5 further comprising a membrane connected to an array of needles wherein the membrane is a flexible material.

7. The adapter of claim 1 wherein the membrane comprises second cavity in fluid communication with the fluid cavity and the microneedle array.

8. A medication delivery pen comprising:
a housing;
a medication cartridge mounted within the housing;
a plunger mountable to the medication cartridge;
a needle mountable to a second portion of the medication cartridge; and
an adapter that receives a syringe mounted to the needle wherein the adapter has a microneedle array.

9. The delivery pen of claim 8 wherein the medication cartridge comprises insulin or growth hormone.

10. The delivery pen of claim 8 wherein the adapter further comprises a septum mounted within the adapter.

11. The delivery pen of claim 8 wherein the adapter further comprises a plurality of fluid channels in fluid communication with the microneedle array.

12. The delivery pen of claim 8 wherein the microneedle array comprises a membrane that mounts to a distal surface of the adapter.

13. The delivery pen of claim 12 wherein the membrane comprises a flexible material.

14. The delivery pen of claim 12 wherein the membrane comprises a second cavity in fluid communication with the fluid cavity and the microneedle array.

15. An infusion set comprising:
a housing;
a fluid conduit housing a first end mounted within the housing and a second end attached to a fluid source;
a needle attached to the housing; and
an adapter mounted to the needle wherein the adapter has a microneedle array.

16. The infusion set of claim 15 wherein the adapter further comprises a septum mounted within the adapter.

17. The infusion set of claim 15 wherein the adapter further comprises a plurality of fluid channels in fluid communication with the microneedle array.

18. The infusion set of claim 15 wherein the microneedle is attached to the housing at an angle in the range between 5° and 60° relative to a long axis of the housing.

19. The infusion set of claim 15 wherein the microneedle array comprises a membrane that mounts to a distal surface of the housing.

20. The infusion set of claim 19 wherein the membrane comprises a flexible material.

21. The adapter of claim 1 wherein the membrane comprises a second cavity in fluid communication with the fluid cavity in the microchannel array.

22. A method for injecting a fluid into an injection site comprising:
providing a fluid coupled with a needle and an adapter having a microneedle array;
placing the microneedle array in fluid communication with the needle;
positioning the microneedle array at an injection site; and
injecting the fluid through the needle and the microneedle array into the injection site.

23. The method of claim 22 further comprising penetrating a seal in the adapter and injecting fluid into a fluid cavity in the adapter.

24. The method of claim 22 further comprising:
removing a syringe and needle from the adapter;
maintaining the microneedle array within the injection site;
placing a second syringe and needle in fluid communication with the microneedle array; and
injecting a fluid from the second syringe through the microneedle array and into the injection site.

25. The method of claim 22 further comprising connecting the needle to a fluid pump.

26. The method of claim 22 further comprising providing an infusion set having an infusion needle and inserting the infusion needle into the adapter.

27. The method of claim 26 further comprising providing a fluid source that is in fluid communication with the infusion needle.

28. The method of claim 26 further comprising providing the microneedle array with a plurality of needles that extend along a first axis and inserting the infusion needle along a second axis having an angle between 10° and 90° relative to the first axis.

29. The method of claim 22 further comprising providing an expandable cavity that enlarges a volume of the cavity during transfer of fluid from the needle into the cavity.

30. The adapter of claim 1, wherein the microneedle device includes at least one microneedle having a length of at least about 500 microns.

31. The medication delivery pen of claim 8, wherein the microneedle array includes at least one microneedle having a length of at least about 500 microns.

32. The infusion set of claim 15, wherein the microneedle array includes at least one microneedle having a length of at least about 500 microns.

33. The method of claim 22, wherein the microneedle array includes at least one microneedle having a length of at least about 500 microns.
